(19) United States
(12) Patent Application Publication
(10) Pub. No.: US 2008/0257351 A1
(43) Pub. Date: Oct. 23, 2008

(54) RESUSCITATION DEVICE

(76) Inventor: David Gitschlag, Colorado Springs, CO (US)

Correspondence Address: BRENDA L. SPEER;BRENDA L SPEER, LLC, 2 NORTH CASCADE AVENUE, SUITE 1100, COLORADO SPRINGS, CO, 80903, US

(21) Appl. No.: 12/106,288

(22) Filed: Apr. 19, 2008

Related U.S. Application Data
(60) Provisional application No. 60/925,086, filed on Apr. 19, 2007.

(51) Int. Cl. A61M 16/00; A62B 18/02; A61F 5/00

(52) U.S. Cl. 128/205.13; 128/205.25; 602/18

(57) ABSTRACT

The device of the present invention relates to a resuscitation device that can be operated by a single user in a hands-free manner and a method of using the resuscitation device for administration by the user to a patient of either pulmonary resuscitation or cardio-pulmonary resuscitation (CPR) which employs fresh air. The device of the present invention may be either human-actuated or non-human actuated. Typically, when used in emergency situations, the device of the present invention is human-actuated. The device of the present invention comprises a neck support means interconnectedly related to a mask; wherein the mask is interconnectedly related to a respiratory hose by means of a one-way valve coupling; and further wherein the respiratory hose is interconnectedly related to a bellows device by means of a one-way valve coupling.

### CROSS-REFERENCE TO RELATED APPLICATION

This application claims priority from provisional patent application Ser. No. 60/925,086, filed Apr. 19, 2007, for Single Person CPR Respiration Device by Gitschlag.

### BACKGROUND OF THE INVENTION

1. Field of the Invention

The device of the present invention relates to a resuscitation device that can be operated by a single user in a hands-free manner and a method of using the resuscitation device for administration by the user to a patient of either pulmonary resuscitation or cardio-pulmonary resuscitation (CPR) which employs fresh air.

2. Description of Related Art Including Information Disclosed Under 37 CFR 1.97 and 37 CFR 1.98

Cardio-pulmonary resuscitation (“CPR”) is a known methodology. CPR may be administered manually without the assistance of any device, such as mouth-to-mouth delivery of pulmonary resuscitation directly from a CPR administrator and to a patient, in conjunction with manual compression by the CPR administrator of the patient's chest to deliver simultaneously cardiac massage or resuscitation. There are numerous prior art devices to assist with the administration of CPR.

Over time, the methods and equipment used for CPR have evolved from direct mouth-to-mouth administration, to mouth-to-mouth administration devices, to device-to-mouth administration devices. In particular, there are numerous prior art devices to assist with the administration of a pulmonary resuscitation element of CPR. For instance, U.S. Pat. No. 2,280,050 issued Feb. 12, 1942, by Alexander et al. for Resuscitator discloses a resuscitator comprising a tube for blowing air into a patient's lungs. A similar device is disclosed in U.S. Pat. No. 3,017,880 issued Nov. 4, 1958, by Brook for Resuscitator which resuscitator also has a suction tube for removing obstructing secretions from the patient's airway. Yet other similar devices are U.S. Pat. No. 3,018,775 issued Jan. 30, 1962, by Wilson et al. for Resuscitator Apparatus; and U.S. Pat. No. 3,099,985 issued Dec. 21, 1960, also by Wilson et al. for Resuscitator Apparatus, both of which disclose a tube for administration of air from a resuscitator into the lungs of a patient.

Some prior art devices provide for protected, self-controlled respiration, such as U.S. Pat. No. 2,850,011 issued Sep. 25, 1956, by Schaefer for Respiratory Helmet that discloses a respiratory helmet comprising a cap which encompasses a user's head and which contains a pressurized breathing mixture.

Other prior art that discloses devices for use in mouth-to-mouth resuscitation, wherein the device serves as an intermediary between a resuscitator and a patient for delivery of pulmonary resuscitation, are: U.S. Pat. No. 2,887,104 issued May 19, 1959, by Sovinsky et al. for Mask to Mask Resuscitator (discloses mask to mask resuscitation in a contaminated atmosphere from a resuscitator to a patient); and U.S. Pat. No. 3,158,152 issued Sep. 16, 1960, by Bloom for Mouth to Mouth Resuscitator (provides for the infusion of fresh air into a patient's lungs via a resuscitator's operation of the device).

Prior art devices which provide for the infusion of air exhaled by a resuscitator into a patient's lungs via an intermediary device are: U.S. Pat. No. 3,219,030 issued Feb. 9, 1962, by Bartlett, Jr., for Apparatus for Use in Mouth-to-Mouth Resuscitation, and U.S. Pat. No. 3,286,710 issued Nov. 22, 1966, also by Bartlett, Jr., for Apparatus for Use in Mouth-to-Mouth Resuscitation; U.S. Pat. No. 3,518,989 issued Feb. 14, 1966, by Seeler for Valve Assembly; U.S. Pat. No. 3,923,054 issued May 13, 1974 by Bauer, Jr., for Resuscitation Device; U.S. Pat. No. 4,106,502 issued Aug. 15, 1978, by Wilson for Resuscitator; and U.S. Pat. No. 4,579,114 issued Apr. 1, 1986, by Gray et al. for Mouth to Mouth Resuscitation Device.

There are numerous prior art devices that allow a user to perform pulmonary resuscitation upon a patient by infusion of air into the patient's lungs through a mechanical device, rather than by mouth-to-mouth, or by a mouth-to-mouth administration device. Such prior art devices are: U.S. Pat. No. 3,046,978 issued Jun. 22, 1960, by Lea for Manually Operated Resuscitator (an air pump in the form of a bladder for forcing air through a nozzle and into a patient's airway); U.S. Pat. No. 3,083,707 issued Feb. 13, 1963, by Seeler for Device for Treatment of Pulmonary Diseases (a pressure control apparatus to deliver a flow of gas under pressure to a patient's lungs); U.S. Pat. No. 3,262,466 issued Jul. 26, 1966, by Stoner for Resuscitator (a manually operable respiration device for forcible injection of air into a patient via a hand bulb); and U.S. Pat. No. 4,513,741 issued Apr. 30, 1985, by Demi for Apparatus Adaptable for Treating Animals (a respiratory device particularly adapted for use with animals).

Other similar prior art devices that allow a user to perform pulmonary resuscitation upon a patient by infusion of air into the patient's lungs through a mechanical device are: U.S. Pat. No. 3,216,413 issued Nov. 9, 1965, by Mota for Portable Artificial Respirator; U.S. Pat. No. 4,029,093 issued Jun. 14, 1977, by Køhnke for Gas Supply Device; U.S. Pat. No. 4,088,131 issued May 9, 1978, by Elam et al. for Breathing Assistance Device; U.S. Pat. No. 4,374,521 issued Feb. 22, 1983, by Nelson et al. for Squeeze Bag Type Resuscitator Apparatus; U.S. Pat. No. 4,501,271 issued Feb. 26, 1985, by Clifton et al. for Resuscitator; and U.S. Pat. No. 4,774,941 issued Oct. 4, 1988, by Cook for Resuscitator Bag.

Essentially all of these prior art devices that allow a user to perform pulmonary resuscitation upon a patient by infusion of air into the patient's lungs through a mechanical device employ an inflatable and squeezable device, such as a bag, bladder, bellows or the like, to draw air into and to expel air from the inflatable, squeezable device through the respiratory device and into the patient's lungs. Examples of such prior art inflatable devices are: U.S. Pat. No. 2,741,402 issued Apr. 10, 1956, by Sayre for Plastic Container with Welded Seam; U.S. Pat. No. 3,473,529 issued Oct. 21, 1969, by Wallace for Squeeze-Bag Resuscitator; and U.S. Pat. No. 5,163,424 issued Nov. 17, 1992, by Køhnke for Disposable Resuscitator.

In prior art respiratory devices, various masks are known which cover a patient's nose and mouth for ease of administration of air into the patient's respiratory system. Examples of such prior art masks used in the administration of pulmonary resuscitation or regulation are: U.S. Pat. No. 3,796,216 issued Mar. 12, 1974, by Schwarz for Resuscitator; and U.S. Pat. No. 4,811,730 issued Mar. 14, 1989, by Milano for CPR Face Mask and Method of Using Same.

In prior art respiratory devices, various valves are known which permit the regulation of air flow into and/or out of a patient's lungs during administration of pulmonary resuscitation or other method of delivery of air or oxygen to a patient. Examples of such prior art devices are: U.S. Pat. No. 3,242,921 issued Mar. 29, 1966, by Seeler for Breathing Control Valve, and U.S. Pat. No. 3,356,100 issued Dec. 5, 1967, also by Seeler for Breathing Control Valve and Operator Therefor (provides a valve for regulation of air flow into and out of a patient's lungs in conjunction with a resuscitation device); U.S. Pat. No. 3,610,236 issued Oct. 5, 1971, by Smilg for Resuscitator Device (resuscitation valve to receive oxygen under pressure and alternately exhaust same to the atmosphere); U.S. Pat. No. 3,827,440 issued Aug. 6, 1974, by Birch et al. for Check-Valve for Tracheotomy Tubes (tracheotomy check-valve operated by a wearer's normal breathing); U.S. Pat. No. 4,077,404 issued Mar. 7, 1978, by Elam for Breathing Equipment Such as Resuscitators (provides a valve for regulation of air flow into and out of a patient's lungs in conjunction with a resuscitation device); U.S. Pat. No. 4,601,465 issued Jul. 22, 1986, by Roy for Device for Stimulating the Human Respiratory System (air flow control device to stimulate the respiratory system by making breathing more difficult and limiting lung ventilation); and European Patent EP 0 139 363 filed Jan. 8, 1984, by Flynn for Breathing Apparatus (provides a valve for regulation of air flow into and out of a patient's lungs in conjunction with a resuscitation device).

Prior art coupling devices are known for connecting pipes, hoses or tubes to other pipes, hoses or tubes; connecting piping, hosing or tubing to a device, and the like. Such coupling devices are commonly employed in pulmonary resuscitation devices to interconnect a bellows device with piping, hosing or tubing and to interconnect the piping, hosing or tubing with a mask for transfer of air to a patient through the resuscitation device upon operation of the bellows. Examples of such prior art devices are: U.S. Pat. No. 693,830 issued Feb. 25, 1902, by Burke for Pipe-Coupling; U.S. Pat. No. 2,489,831 issued Oct. 18, 1947, by Veitch for Pipe Joint; U.S. Pat. No. 3,224,795 issued Dec. 21, 1965, by Conley for Flanged Fitting with a Reinforcing Sleeve; U.S. Pat. No. 3,264,013 issued Aug. 2, 1966, by Richardson et al. for Coupling for Pipe Sections; U.S. Pat. No. 3,473,833 issued Oct. 21, 1969, by Bremer for Plastic Pipe Coupling; U.S. Pat. No. 3,654,965 issued Apr. 11, 1972, by Gramain for Closure Members for Pipe Sections; U.S. Pat. No. 3,726,320 issued Apr. 10, 1973, by Lachenmayer for Tubular Construction Arrangement; and U.S. Pat. No. 3,920,787 issued Nov. 18, 1975, by McDowell et al. for Joint Between Tubular Plastic Articles and Method of Forming.

The primary and major disadvantage of all of these prior art resuscitation devices is that none of the prior art devices provide a desirable, efficient and effective means for single user, hands-free operation during administration of CPR, in particular administration of CPR in which fresh air is transfused into a patient's lungs, which is preferable. The prior art devices either: (a) transfuse a resuscitator's air, rather than fresh air, into a patient's lungs by means of direct mouth-to-mouth or device-to-mouth transfusion; or (b) transfuse fresh air into a patient's lungs by device-to-mouth transfusion, but not in a manner that permits single user and/or hands-free operation and delivery of CPR.

### BRIEF SUMMARY OF THE INVENTION

The device of the present invention relates to a resuscitation device that can be operated by a single user in a hands-free manner and a method of using the resuscitation device for administration by the user to a patient of either pulmonary resuscitation or cardio-pulmonary resuscitation (CPR) which employs fresh air. The device of the present invention may be either human-actuated or non-human actuated. Typically, when used in emergency situations, the device of the present invention is human-actuated. The device of the present invention comprises a neck support means interconnectedly related to a mask; wherein the mask is interconnectedly related to a respiratory hose by means of a one-way valve coupling; and further wherein the respiratory hose is interconnectedly related to a bellows device by means of a one-way valve coupling.

### BRIEF DESCRIPTION OF THE SEVERAL VIEWS OF THE DRAWING(S)

FIG. 1 is a side view of a specially fit neck support and a mask as interconnectedly related to a respiratory hose of the device of the present invention.

FIG. 2 is a side view of a bellows of the device of the present invention.

FIG. 3 is a side view of the device of the present invention in use.

### LIST OF REFERENCE NUMERALS

2 device
4 neck support means
6 strap
8 strap coupling means
10 mask
12 mask one-way valve coupling
14 respiratory hose
16 bellows one-way valve coupling
18 bellows
20 handle
22 mask air exhaust means
24 bellows air intake means
26 air reservoir
28 oxygen hose
30 bypass means
32 user
34 patient

### DETAILED DESCRIPTION OF THE INVENTION

The device of the present invention is preferable to the prior art resuscitation devices, because the device of the present invention provides a single user, hands-free operable, desirable, efficient, effective and rapidly deployed means for administration of CPR, in particular administration of CPR in which fresh air is transfused into a patient's lungs, which is preferable. The fresh air may be either atmospheric air or direct air from a supply source, such as from an oxygen tank.

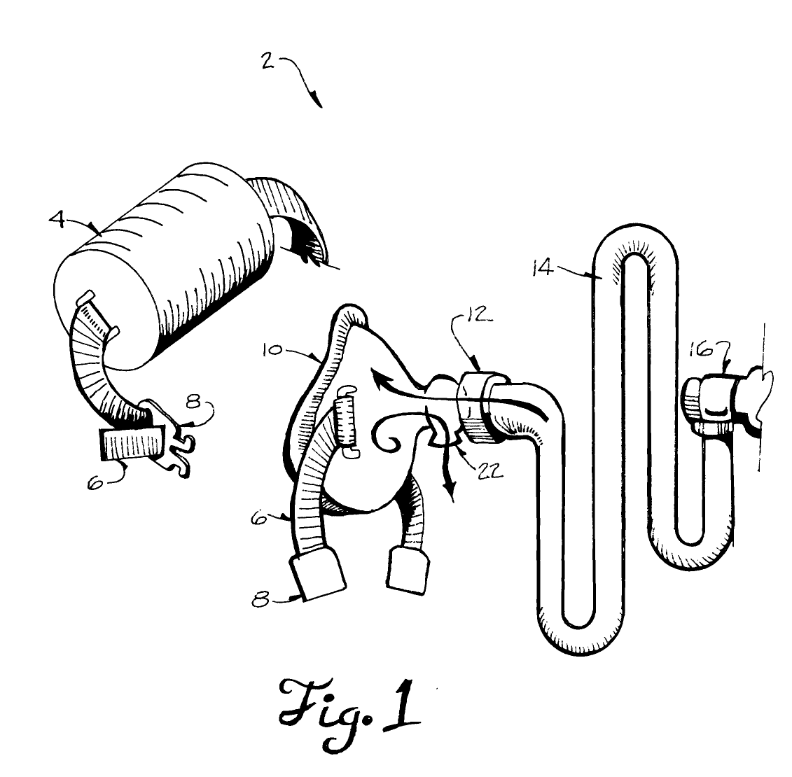

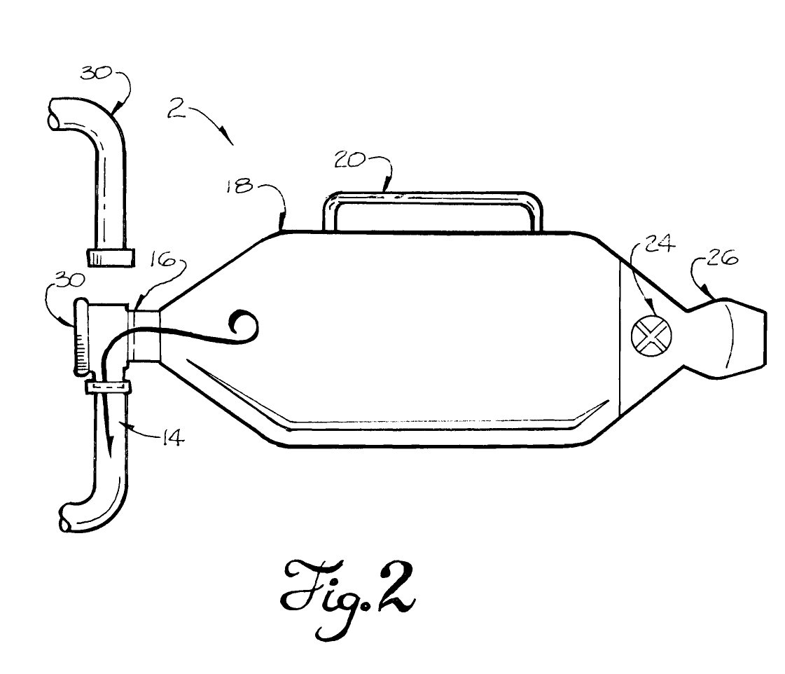

With reference to FIG. 1 and FIG. 2, the device 2 of the present invention comprises a mask 10; wherein the mask 10 is interconnectedly related to a respiratory hose 14 by means of a one-way valve coupling 12; and further wherein the respiratory hose 14 is interconnectedly related to a bellows 18 by means of a one-way valve coupling 16. The device 2 of the present invention further comprises a patient neck support means 4 interconnectedly related to the mask 10 by means of a strap 6.

The neck support means 4 of the device 2 of the present invention may be any neck support means 4 which provides support to the neck and/or head of a patient during administration of CPR; wherein the neck support means 4 is supportive, stable and semi-rigid, yet sufficiently deformable to receive and cradle, preferably stably cradle, a neck and/or head of the patient, and which configures and maintains the patient in a position in which the patient's trachea, or airway, remains open for inspiration and exhalation by the patient. As an illustrative, non-limiting example, the neck support means 4 may comprise a pillow made of foam, either as a unitary block or as filler material; or a pillow made of a casing stuffed with any suitable filler material. Preferably, the neck support means 4 has a removable cover such that it may be reused in compliance with medical regulations. Alternatively, the neck support means 4 may be readily disposable and replaceable for compliance with medical regulations.

The mask 10 of the device 2 of the present invention may be any suitable mask 10 known in the art to be suitable for respiratory purposes. Preferably, the mask 10 of the device 2 of the present invention covers a nose and mouth of a patient. The mask 10 also preferably is sufficiently deformable to create an airtight seal around the patient's nose and mouth. The mask 10 also preferably is made of a transparent material to allow the user to visually determine if there is any obstruction in the patient's airway, which in turn eliminates the need to temporarily stop the administration of CPR to perform such a check by removal of the mask 10 from the patient's face.

With reference to FIG. 1, to allow for a proper fit and to aid in the seal of the mask 10 around the patient's nose and mouth, the mask 10 is interconnectedly related to a strap 6 or straps 6. In turn, the straps 6 are interconnectedly related to the neck support means 4. Preferably, the straps 6 are fitted with any suitable strap coupling means 8 which allows for the rapid attachment and detachment of the neck support means 4 in interconnected relationship with the mask 10 upon the patient, as well as an adjustment means for adjustment of the straps 6 to a desired length and tension as may be necessary to ensure a tight seal of the mask 10 upon the patient. By way of illustration only, such a suitable strap coupling means 8 may be quick release clips as shown in FIG. 1 having interlocking male and female coupling components; further wherein the quick release clips have an adjustment means by which to allow for the tightening and slackening of the straps 6 as necessary, such as a ladder rung mechanism in which a strap 6 is threaded through and then rethreaded back through back upon and in opposing relationship to itself.

An advantage of the device 2 of the present invention over the prior art is that it does not need to be removed for or during transport of a patient, such as to a treatment facility. A further advantage of the device 2 of the present invention over the prior art is that the strap coupling means 8 allows for the rapid attachment and detachment of the neck support means 4 about the patient's neck and the mask 10 about the patient's face, to properly position the patient for administration of CPR, or, if necessary, to clear the patient's airway, to allow the patient to vomit, for intubation of the patient, and the like.

The mask 10 is interconnectedly related to a proximal end of a respiratory hose 14 by means of a one-way valve coupling 12. As shown by the directional arrows in FIG. 1, the one-way valve coupling means 12 allows for uni-directional flow of air from the respiratory hose 14 into the mask 10 for inspiration by the patient. The mask 10 also has a mask air exhaust means 22 which allows for unidirectional evacuation of air exhaled by the patient from the mask 10 into the atmosphere as shown by the directional arrows.

The respiratory hose 14 is any suitable length to allow for operation of the device of the present invention, but preferably is approximately two feet (75 cm) in length. A distal end of the respiratory hose 14 is interconnectedly related to a bellows 18 by means of a bellows one-way valve coupling 16.

The respiratory hose 14 and an oxygen hose 28 of the device of the present invention may be any suitable piping, hosing or tubing known in the art. Preferably the hoses 14, 28 are made of a durable, yet sufficiently flexible material to allow for coiling of the hoses 14, 28 for storage purposes, space-saving purposes, ease of transport of the device 2 and the like.

With reference to FIG. 2, the device 2 of the present invention further comprises a bellows 18 to pump air through the respiratory hose 14 and into the mask 10 for inspiration by the patient during the administration of pulmonary resuscitation. The bellows 18 may be any suitable device known in the art that allows for air to be pumped through the device 2 of the present invention. As shown in FIG. 2, by way of illustration only, the bellows 18 may be an inflatable, squeezable bag or bladder. The bellows 18 has a bellows air intake means 24. The bellows air intake means 24 allows for the unidirectional intake of air from the atmosphere into the bellows 18 for inflation of the bellows 18. Any suitable one-way valve known in the art may serve as the bellows air intake means 24.

As shown in FIG. 2, the bellows 18 optionally further comprises an air reservoir 26 to allow for sufficient inflation and deflation of the bellows 18 when operated by a user.

Also as shown in FIG. 2, the bellows 18 optionally further comprises a bypass means 30 which permits the bellows 18 to be interrelatedly connected to a direct air supply source, such as from an oxygen tank. In this alternative embodiment of the device 2 of the present invention, the bellows 18 may be interconnectedly related to a proximal end of an oxygen hose 28 at either the bypass means 30, or the bellows air intake means 24. In turn, a distal end of the oxygen hose 28 may be interconnectedly related to a direct air supply source, such as oxygen from a pressurized air tank, to allow delivery of fresh air to a patient by, as necessary, either user-operated, or non-user, automatically operated, use of the device 2 of the present invention. Typically, if the patient is not breathing on his own, then the device 2 of the present invention is user-operated; and it the patient is breathing on his own, then the device 2 of the present invention is non-user, automatically operated.

As further shown in FIG. 2, the bellows 18 of the device 2 may have an optional handle 20. The handle 20 may be used for leverage-assisted or manual compression of the bellows 18 and for ease of transport of the device 2.

Preferably, one-way valves are used for the device 2 of the present invention. One-way valves prevent the transfer and exchange of the patient's bodily gases and fluids (air, blood, mucus, vomitus and the like) among the components of the device 2 of the present invention. Accordingly, the user of the device 2 of the present invention is not exposed to the potentially hazardous, unpleasant and undesirable transfer and exchange of such bodily gases and fluids as occurs in most of the various known prior art methods of CPR. One-way valves also control the direction of air flow through the device 2 of the present invention, namely, as shown in FIG. 1 and FIG. 2, uni-directionally into the bellows 18 through the bellows air intake means 24; uni-directionally from the bellows 18 into the respiratory hose 14 as indicated by the directional arrows; uni-directionally from the respiratory hose 14 into the mask 10 as indicated by the directional arrows for inspiration by the patient; and uni-directionally out of the mask 10 through the mask air exhaust means 24.

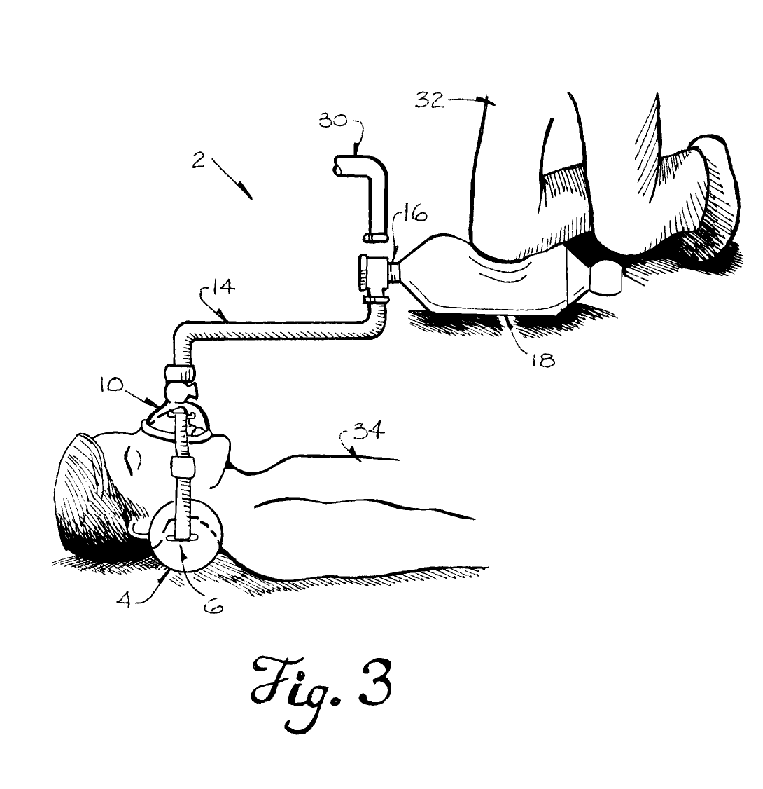

With reference to FIG. 1 and FIG. 2, to use the device 2 of the present invention, the device 2 is assembled by interrelatedly connecting the mask 10 to the respiratory hose 14 by means of the mask one-way valve coupling 12, and the respiratory hose 14 to the bellows 18 by means of the bellows one-way valve coupling 16. With reference to FIG. 3, to administer CPR with the device 2 of the present invention, a user 32 places a patient 34 in a supine position, places the neck support means 4 underneath the neck and/or head of the patient 34, places the mask 10 over a nose and mouth of the patient 34, and interconnectedly relates the neck support means 4 to the mask 10 with the straps 6 and the strap coupling means 8. The user adjusts the strap coupling means 8 if and as needed to create a seal between the mask 10 and the patient's face.

The user 32 places the bellows 18 on the ground near the patient 34 and, preferably, kneels on the bellows 18 with one knee. To operate the bellows 18, the user 32 raises his knee to allow the bellows 18 to inflate with air, then kneels or presses his knee into the bellows 18 to compress the bellows 18 thereby expelling the air from the bellows 18 through the bellows one-way valve coupling 16, into the respiratory hose 14 and through the mask one-way valve coupling 12, and into the mask 8 for either forcible or voluntary inspiration by the patient 34, as the circumstance may be. Alternatively, the user 32 may operate the bellows 18 by compressing the bellows 18 with his hand or hands, either with or without the aid of the handle 20; by placing the bellows 18 under his upper arm in an area between his elbow and axilla and against the side of his torso, and compressing the bellows 18 with his upper arm against his torso; by placing the bellows 18 under his foot and compressing the bellows 18 by stepping on it; or by any other physical, human-actuated, suitable means of compressing the bellows 18. The user 32 cyclically continues to inflate and compress the bellows 18 as needed for administration of CPR to the patient 34.

In an alternative embodiment for use of the device 2 of the present invention, the bypass means 30 of the bellows 18 is interrelatedly connected to a direct air supply source by means of an oxygen hose 28. In this alternative embodiment, rather than the user 32 operating of the bellows 18 to deliver fresh air to the patient 34, fresh air is automatically delivered to the patient 34 from the direct air supply source.

Simultaneously with or after the user 32 has commenced pulmonary resuscitation administration to the patient 34 with the device 2 of the present invention, the user 32 begins administration of cardiac massage or resuscitation through compressions of a chest of the patient 34 in accordance with CPR methodologies known in the art. After the user 32 has administered the desired number of chest compressions to the patient 34, the user 32 then either continues or momentarily ceases chest compressions, and cyclically inflates and compresses the bellows 18, by any suitable means such as by lifting his knee from and pressing his knee into the bellows 18, as many times as needed for the administration of pulmonary resuscitation to the patient 34 as previously described.

In contrast to the prior art one- and/or two-handed CPR methodologies and devices, use of the device 2 of the present invention to administer CPR to the patient 34 allows the user 32 to operate the device 2 in a single user, hands-free manner, thus leaving the user's 32 hands free to execute other functions, such as checking the patient's pulse, staunching a flow of blood from a wound, and the like, while the user 32 continues to administer CPR to the patient 34 as needed. Other attendant advantages of the device 2 of the present invention over the prior art are set forth below.

The patient's 34 airway will remain open without the continuing aid of the user 32, which in turn allows the user 32 to deliver cardiac massage or resuscitation to the patient 34 without the need for assistance from third parties, and allows the patient 34 to breath unaided, either on his own or with the assistance of the device 2 of the present invention.

The administration of CPR by the user 32 to the patient 34 with the device 2 of the present invention decreases a time interval between a pulmonary cycle and a compression cycle of CPR, thereby greatly diminishing and possibly eliminating the interruption between said cycles. Furthermore, the administration of CPR to a patient by a user with the device 2 of the present invention may be administered in a manner that most closely resembles a patient's normal breathing and heartbeat rhythms, which average from about four to about seven heartbeats to one inspiration and exhalation breathing cycle.

With the device 2 of the present invention, the user 32 can easily sustain and maintain the administration of CPR to a patient 34 for an extended period of time. For instance, the user 32 does not have to perform mouth-to-mouth or device-to-mouth CPR in accordance with prior art methodologies. As a result, the user 32 does not have to use his own air supply to perform pulmonary resuscitation upon the patient 34, which enables the user to administer CPR to the patient 34 with minimal exertion for an extended period of time. Additionally, the device 2 of the present invention enables the user 32 to provide the patient 34 with air having a higher concentration of oxygen during pulmonary resuscitation through the use of atmospheric air provided through operation of the bellows 18, rather than with oxygen-concentration diminished and/or depleted air exhaled by a user and into a patient, such as during performance of mouth-to-mouth or device-to-mouth CPR.

Commonly, a user fears being contaminated with a disease, or the realistic fear of the patient vomiting on a user or in a user's mouth while performing CPR on a patient. The device 2 of the present invention serves to prevent mouth-to-mouth contact, thereby, circumventing exposure to potentially hazardous body fluids.

As a result of the attendant advantages herein described of the device 2 of the present invention, administration of CPR with the device 2 of the present invention provides for the achievement of an increased survival rate for a patient as compared to administration of CPR by other prior art methodologies.

The device 2 of the present invention may be used in any emergency situation where CPR may be required. The light weight of the device 2 and the ability to contain the device 2 in a small area or compartment make the device 2 ideal for placement into emergency vehicles such as police cars, fire engines, ambulances, or any other emergency or personal motor craft, such as boats, planes and the like, for ready access and onsite deployment of the device 2.

Another opportunity for ready access and onsite deployment of the device 2 is by military personnel, either by a troop or a medic. The compact size of the device 2 also makes it suitable to be carried by military personnel in a backpack, vehicle or the like.

The device 2 of the present invention is also suitable to be carried in private commercial or civilian vehicles, planes or water craft, and is easily operated by civilians or persons trained in first aid administration.

The device 2 of the present invention is also suitable for use by mountain rescue and forest service personnel. There are many areas where the terrain does not allow for heavy rescue devices, but the device 2 of the present invention could be easily placed in a rescue pack.

Another advantage of the device 2 of the present invention is that the device 2 simplifies and streamlines CPR training of a user, because a user need not be trained in other, decreased efficiency, prior art CPR methodologies. Yet another advantage of the device 2 of the present invention is that the device 2 may be readily adapted for use with any person, adult or child, regardless of a person's size, through adjustment of the strap 6 as previously described to ensure a seal between the mask 10 and a patient's face, and/or use of a mask of a size most appropriate for a particular patient.

Although the present invention has been described with reference to specific embodiments, modifications and variations of the present invention are possible without departing from the scope of the invention, which is defined by the claims set forth below.

The invention claimed is:

1. A resuscitation device comprising:
a. A neck support means, wherein said neck support means is interconnectedly related to:
b. A mask, wherein said mask is interconnectedly related to:
c. A respiratory hose, wherein said respiratory hose is interconnectedly related to:
d. A bellows; and
further wherein the device is operable by a single user in a hands-free manner; and
further wherein the device permits administration by a user to a patient of pulmonary resuscitation with fresh air.

2. The resuscitation device of claim 1 further wherein the neck support means is interconnectedly related to the mask by at least one strap.

3. The resuscitation device of claim 2 further wherein the strap further comprises a strap coupling means which permits connection of the strap to another strap and adjustment of the strap a desired length and tension.

4. The resuscitation device of claim 1 further wherein the mask is interconnectedly related to the respiratory hose by a one-way valve coupling means.

5. The resuscitation device of claim 4 further wherein the one-way valve coupling means permits air flow only in a direction from the respiratory hose into the mask.

6. The resuscitation device of claim 1 further wherein the mask comprises at least a mask air exhaust means.

7. The resuscitation device of claim 1 further wherein the respiratory hose is interconnectedly related to the bellows by a one-way valve coupling means.

8. The resuscitation device of claim 7 further wherein the one-way valve coupling means permits air flow only in a direction from the bellows into the respiratory hose.

9. The resuscitation device of claim 1 further wherein the bellows comprises at least a bellows air intake means.

10. The resuscitation device of claim 1 further wherein the bellows has an air reservoir.

11. The resuscitation device of claim 1 further wherein the bellows further comprises a bypass means which permits interrelated connection of the bellows with a direct air supply source.

12. A resuscitation device comprising:
a. A neck support means, wherein the neck support means is interconnectedly related to a mask by at least one strap; further wherein the strap further comprises a strap coupling means which permits connection of the strap to a strap of a mask and adjustment of the straps to a desired length and tension;
b. A mask, wherein said mask is interconnectedly related to a respiratory hose by a one-way valve coupling means; further wherein the one-way valve coupling means permits air flow only in a direction from the respiratory hose into the mask; further wherein the mask comprises at least a mask air exhaust means;
c. A respiratory hose, wherein said respiratory hose is interconnectedly related to a bellows by a one-way valve coupling means; further wherein the one-way valve coupling means permits air flow only in a direction from the bellows into the respiratory hose; and
d. A bellows, wherein said bellows further comprises at least a bellows air intake means and at least an air reservoir; and
further wherein the device is operable by a single user in a hands-free manner; and
further wherein the device permits administration by a user to a patient of pulmonary resuscitation with fresh air.

13. The resuscitation device of claim 12 further wherein the bellows further comprises a bypass means which permits interrelated connection of the bellows with a direct air supply source.

14. A method of administering pulmonary resuscitation by a user to a patient comprising the steps of the user:
a. Using a resuscitation device comprising a neck support means, wherein said neck support means is interconnectedly related to a mask, wherein said mask is interconnectedly related to a respiratory hose, wherein said respiratory hose is interconnectedly related to a bellows; further wherein the device is operable by a single user in a hands-free manner; and further wherein the device permits administration by a user to a patient of pulmonary resuscitation with fresh air;
b. Placing the patient in a supine position;
c. Placing the neck support means underneath the neck and/or head of the patient;
d. Placing the mask over a nose and mouth of the patient; and
e. Delivering fresh air to the patient by operating the bellows.

15. The method of claim 14 further wherein the step of delivering fresh air further comprises the user operating the bellows by kneeling on the bellows with one knee, raising his knee to allow the bellows to inflate with air, pressing his knee into the bellows to compress the bellows, thereby expelling the air from the bellows into the respiratory hose and into the mask for inspiration by the patient.

16. The method of claim 15 further comprising, after the step of placing the mask and before the step of delivering fresh air, the step of ensuring a seal between the mask and the patient's face.

17. The method of claim 16 further wherein the step of ensuring a seal between the mask and the patient's face comprises adjusting a strap of the neck support means and a strap of the mask, wherein the neck support means strap and the mask strap are interconnectedly related, further wherein the straps further comprise a strap coupling means which connects the neck support means strap to the mask strap and which allows the user to adjust the straps to a desired length and tension to ensure said seal.

18. A method of administering pulmonary resuscitation by a user to a patient comprising the steps of the user:
a. Using a resuscitation device comprising a neck support means, wherein said neck support means is interconnectedly related to a mask, wherein said mask is interconnectedly related to a respiratory hose, wherein said respiratory hose is interconnectedly related to a bellows; further wherein the device is operable by a single user in a hands-free manner; and further wherein the device permits administration by a user to a patient of pulmonary resuscitation with fresh air;
b. Placing the patient in a supine position;
c. Placing the neck support means underneath the neck and/or head of the patient;
d. Placing the mask over a nose and mouth of the patient;
e. Delivering fresh air to the patient from a direct air supply source, further wherein the bellows comprises a bypass means which permits interrelated connection with the direct air supply source.

19. The method of claim 18 further comprising, after the step of placing the mask and before the step of delivering fresh air, the step of ensuring a seal between the mask and the patient's face.

20. The method of claim 19 further wherein the step of ensuring a seal between the mask and the patient's face comprises adjusting a strap of the neck support means and a strap of the mask, wherein the neck support means strap and the mask strap are interconnectedly related, further wherein the straps further comprise a strap coupling means which connects the neck support means strap to the mask strap and which allows the user to adjust the straps to a desired length and tension to ensure said seal.
